(19) United States
(12) Patent Application Publication
(10) Pub. No.: US 2019/0330180 A1
(43) Pub. Date: Oct. 31, 2019

(54) PROCESS FOR THE PREPARATION OF HERBICIDAL PYRIDINYLIMIDAZOLONE COMPOUNDS

(71) Applicant: SYNGENTA PARTICIPATIONS AG, Basel (CH)

(72) Inventors: Helmars SMITS, Münchwilen (CH); Sujit Kumar GHORAI, Corlim Ilhas (IN)

(73) Assignee: SYNGENTA PARTICIPATIONS AG, Basel (CH)

(21) Appl. No.: 16/097,106

(22) PCT Filed: Apr. 24, 2017
(86) PCT No.: PCT/EP2017/059620

§ 371 Date: Oct. 26, 2018

(30) Foreign Application Priority Data
Apr. 29, 2016 (IN) 201611015026

(51) Int. Cl. C07D 401/04; C07D 213/75; C07D 263/26

(52) U.S. Cl. CPC C07D 401/04; C07D 263/26; C07D 213/75

(57) ABSTRACT

The present invention relates to a process for the preparation of a compound of formula wherein R¹, R², R³, R⁴, R⁵ and R⁶ are as defined in the specification.

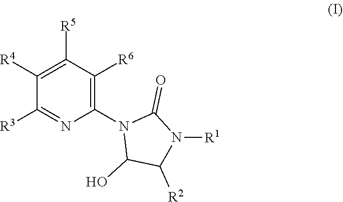

The present invention relates to the preparation of pyridinylimidazolones of formula (I)

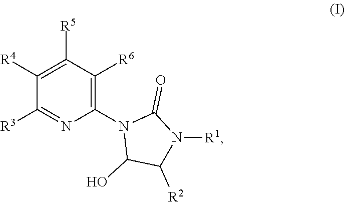

wherein R¹ is selected from C₁-C₆ alkyl, C₃-C₆ cycloalkyl, C₁-C₆ alkoxy and aryl, R² is selected from C₁-C₆ alkyl and hydrogen and R³, R⁴, R⁵ and R⁶ are each independently selected from hydrogen, C₁-C₆ alkyl, C₁-C₆ haloalkyl, nitro and halogen.

Pyridinylimidazolones of general formula (I) are known to be herbicidally active as described in WO 2015/059262, WO 2015/052076 and U.S. Pat. No. 4,600,430.

Methods of preparing compounds of formula (I) are described in U.S. Pat. No. 4,600,430 and WO 2015/059262. The present invention offers unique methods to prepare such compounds using less process steps (presenting therefore advantages such as higher throughput capacity and lower amount of waste) as well as more attractive conditions (for example avoiding the use of ozone or having phenol as a side product). Further, the present invention is suitable for commercial scale production.

It has been described (WO 2014/022116) that pyridine activated as phenyl carbamate could be coupled efficiently with an unprotected N-alkyl amino alcohol to provide a hydroxy urea which would then only need to be oxidized to compounds of formula (I) (Scheme 1). Such an approach is already an improvement over previously described approaches however it is still not satisfactory due to the need to prepare activated pyridine and separate a phenol side product after the coupling step.

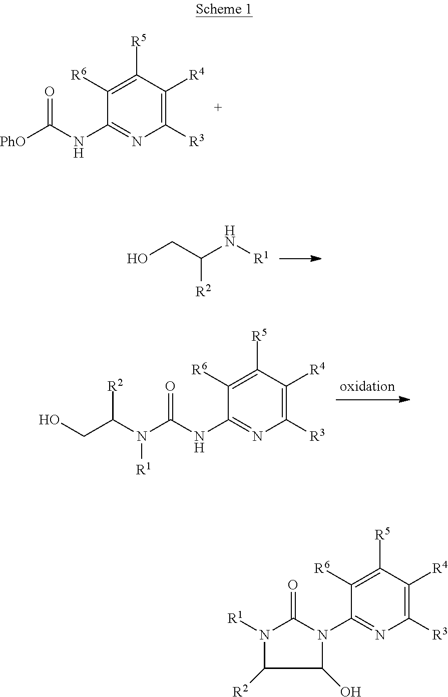

Surprisingly, it has now been found that compounds of formula (II) can be coupled with compounds of formula (III) in the presence of base giving directly compounds of formula (IV) in a highly selective and atom efficient manner. Compounds of formula (IV) are then oxidized to compounds of formula (I) (Scheme 2).

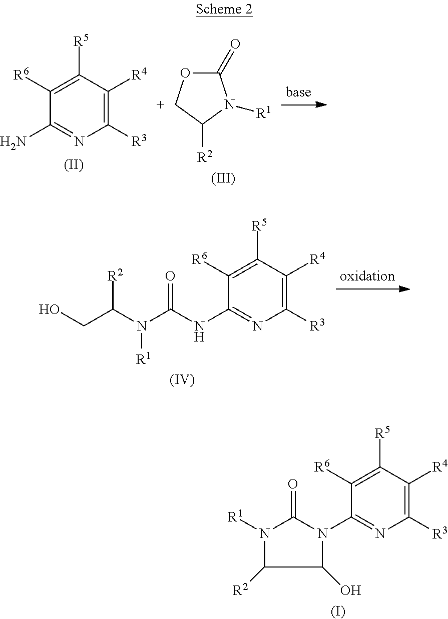

Such reactivity is highly unusual since normally nitrogen nucleophiles upon heating react preferentially at C-5 position of compounds of formula (III) as for example described in Morita, Y.; Ishigaki, T.; Kawamura, K.; Iseki, K. Synthesis 2007, 2517. An intermolecular reaction of nitrogen nucleophiles at C-2 position has been reported only when R¹ is hydrogen (Gabriel, S.; Eschenbach, G. Chem. Ber. 1987, 30, 2494; JP 2014/062071) or an electron withdrawing group (for example as described in Romanenko, V. D.; Thoumazet, C.; Lavallo, V.; Tham, F. S.; Bertrand, G. Chem. Comm. 2003, 14, 1680). In the former case the reaction could also proceed via isocyanate as an intermediate which is not possible when R¹ is not hydrogen. The key parameter of the process of the present invention is a base sufficiently strong to at least partly deprotonate amino group of compound of formula (II) with the driving force of the condensation then being the formation of a less basic anion of compound of formula (IV). The reaction may be an equilibrium process and a slight excess of either compound of formula (II) or compound of formula (III) may be required to drive the reaction to completion.

Thus, according to the present invention, there is provided a process for the preparation of compound of formula (I)

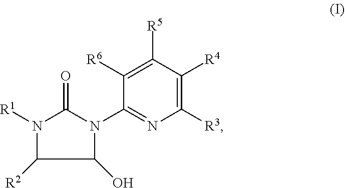

wherein
R¹ is selected from C₁-C₆ alkyl, C₃-C₆ cycloalkyl, C₁-C₆ alkoxy and aryl;
R² is selected from C₁-C₆ alkyl, aryl and hydrogen
R³, R⁴, R⁵ and R⁶ are each independently selected from hydrogen, C₁-C₆ alkyl, C₁-C₆ haloalkyl, nitro and halogen; comprising
a) reacting the compound of formula (II)

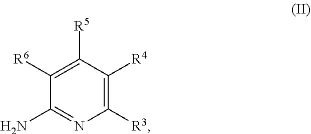

wherein R³, R⁴, R⁵ and R⁶ are as defined above with a strong base and a compound of formula (III)

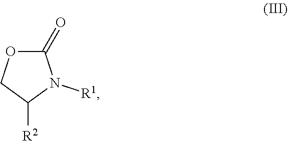

wherein R¹ and R² are as defined above to a compound of formula (IV)

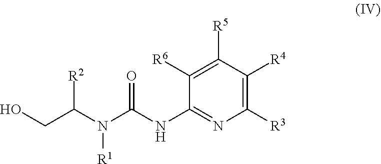

wherein R¹, R², R³, R⁴, R⁵ and R⁶ are as defined above; and
b) reacting the compound of formula (IV) with an oxidizing agent to produce a compound of formula (I)

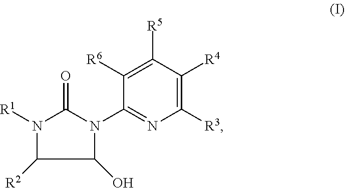

wherein
R¹, R², R³, R⁴, R⁵ and R⁶ are as defined above.

Conveniently, the compounds of formula (III) are prepared by reacting an amino alcohol of formula (V)

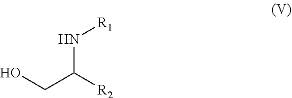

wherein R¹ and R² are as defined above for the compound of formula (I) with a dialkyl carbonate in the presence of base.

In particularly preferred embodiments of the invention, preferred groups for R¹, R², R³, R⁴, R⁵ and R⁶, in any combination thereof, are as set out below.

Preferably, R¹ is selected from C₁-C₅ alkyl and C₁-C₅ alkoxy. More preferably R¹ is selected from methyl and methoxy. More preferably, R¹ is methyl.

Preferably R² is selected from hydrogen and C₁-C₅ alkyl. More preferably, R² is selected from methyl and hydrogen. More preferably R² is hydrogen.

Preferably R³ is selected from hydrogen, C₁-C₄ alkyl, C₁-C₄ haloalkyl and halo. More preferably, R³ is selected from hydrogen, chloro, methyl, difluoromethyl and trifluoromethyl. More preferably, R³ is selected from hydrogen and trifluoromethyl. More preferably R³ is hydrogen.

Preferably R⁴ is selected from hydrogen, C₁-C₄ alkyl, C₁-C₄ haloalkyl and halo. More preferably, R⁴ is selected from hydrogen, chloro, methyl, difluoromethyl and trifluoromethyl. More preferably, R⁴ is selected from hydrogen, chloro and trifluoromethyl and, more preferably, R⁴ is hydrogen.

Preferably R⁵ is selected from hydrogen, C₁-C₄ alkyl, C₁-C₄ haloalkyl and halo. More preferably, R⁵ is selected from hydrogen, chloro, methyl, difluoromethyl and trifluoromethyl. More preferably, R⁵ is selected from hydrogen, methyl and trifluoromethyl and, more preferably, R⁵ is trifluoromethyl.

Preferably R⁶ is selected from hydrogen, C₁-C₄ alkyl, C₁-C₄ haloalkyl and halo. More preferably, R⁶ is selected from hydrogen, chloro, methyl, difluoromethyl and trifluoromethyl. More preferably, R⁶ is hydrogen.

The following scheme 3 describes the reactions of the invention in more detail. The substituent definitions are the same as defined above. The starting materials as well as the intermediates may be purified before use in the next step by state of the art methodologies such as chromatography, crystallization, distillation and filtration.

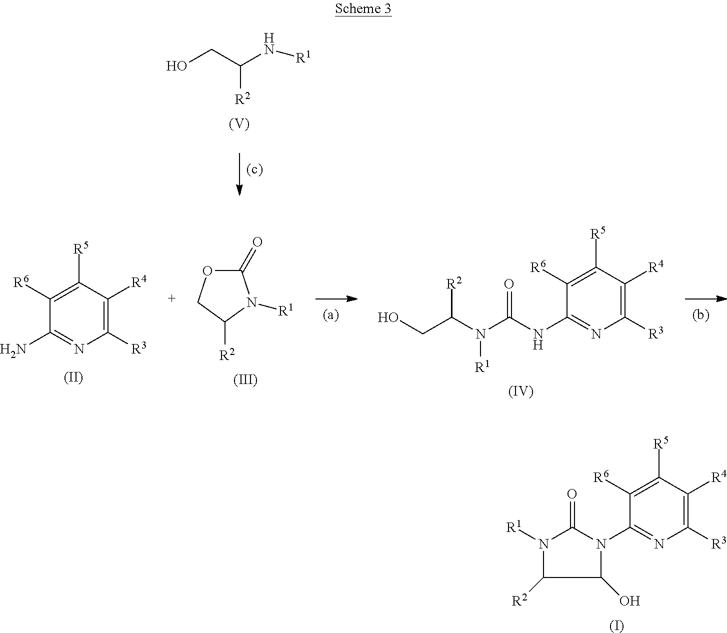

### Step (a):

The compound of formula (IV) can be advantageously prepared by reacting a compound of formula (II) with a base sufficiently strong to deprotonate at least partly the amino group and a compound of formula (III). The strength of the base required is dependent on pKa of compound of formula (II). Suitable bases include, but are not limited to alkali metal alkoxides (such as sodium methoxide, sodium t-butoxide, potassium t-butoxide and sodium ethoxide), alkali metal amides (such as sodium amide, potassium amide, sodium hexamethyldisilazide and potassium hexamethyldisilazide), organolithium reagents (such as n-butyl lithium) and sodium hydride.

The reactions between compounds of formula (II) and (III) are preferably carried out in the presence of a solvent. Suitable solvents include, but are not limited to non-protic organic solvents such as tetrahydrofuran, 2-methyl tetrahydrofuran, t-butyl methyl ether, cyclohexane, toluene, xylenes, acetonitrile and dioxane. The most preferred solvents are tetrahydrofuran, 2-methyl tetrahydrofuran, xylene and toluene.

The reaction can be carried out at a temperature from −20° C. to 100° C., preferably from 10° C. to 50° C. (e.g. no lower than −20° C., preferably no lower than 10° C.; e.g. no more than 100° C., preferably no more than 50° C.).

Aminopyridines of formula (II), where not commercially available, may be made by literature routes such as below and as detailed in J. March, Advanced Organic Chemistry, 4^{th} ed. Wiley, New York 1992.

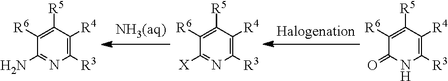

Suitable conditions for effecting these transformations are set out in J. March, Advanced Organic Chemistry, 4^{th} ed. Wiley, New York 1992.

The compounds of formula (III) may be commercially available. When not commercially available the compound of formula (III) can be advantageously prepared by reacting a compound of formula (V) with a dialkyl carbonate in the presence of base as described in more detail in step (c).

### Step (b)

The compound of formula (I) can be advantageously prepared by reacting a compound of formula (IV) with an oxidizing agent. In principle any oxidation reagent known to a person skilled in the art for oxidation of primary alcohols to aldehydes could be employed. Suitable oxidizing agents include, but are not limited to, aqueous sodium hypochlorite, oxygen, Dess-Martin periodinane and dimethylsulfoxide in a presence of an activating agent. When sodium hypochlorite is used, it is preferable to use it in the presence of catalytic amounts of a stable radical such as (2,2,6,6-tetramethylpiperidin-1-yl)oxyl (TEMPO), 4-hydroxy-TEMPO or 4-acetylamino-TEMPO. When dimethylsulfoxide is used, either oxalyl chloride (Swern oxidate) or pyridine sulfur trioxide complex (Parikh-Doering oxidation) can be used as an activating agent. Preferably, the oxidant is an aqueous solution of sodium hypochlorite, most preferably in the presence of catalytic amounts of a stable radical (2,2,6,6-tetramethylpiperidin-1-yl)oxyl (TEMPO), 4-hydroxy-TEMPO or 4-acetylamino-TEMPO. Optionally catalytical amounts of sodium bromide are also added.

The amount of TEMPO based catalysts is between 0.01 and 0.10 equivalents, more preferably between 0.02 and 0.05 equivalents. If sodium bromide is used then the optimal amount is between 0.02 and 0.30 equivalents, more preferably between 0.05 and 0.15 equivalents.

The oxidation of compound (IV) to compound (I) is preferably carried out in the presence of a solvent. Suitable solvents include, but are not limited to, polar non-water miscible solvents such as ethyl acetate, dichloromethane, t-butyl methyl ether, 2-methyl tetrahydrofuran, 1,2-dichloroethane, methyl isobutyl ketone, toluene, chlorobenzene and chloroform. The most preferred solvents are ethyl acetate, toluene and chlorobenzene.

The reaction can be carried out at a temperature from −10° C. to 100° C., preferably from 0° C. to 50° C. (e.g. no lower than −10° C., preferably no lower than 0° C., e.g. no more than 100° C., preferably no more than 50° C.).

### Step (c)

Conveniently, compounds of formula (III) can be prepared by reacting an amino alcohol of formula (V)

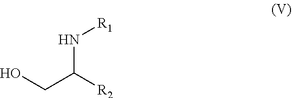

wherein R¹ and R² are as defined above with a dialkyl carbonate in the presence of base as for example described in Vani, P. V. S. N.; Chida, A. S.; Srinivasan, R.; Chandrasekharam, M.; Singh, A. K. Synth. Comm. 2001, 31, 2043.

Typically, the dialkyl carbonate is a C₁-C₆ dialkyl carbonate, such as dimethyl carbonate and diethyl carbonate. Suitable bases include, but are not limited to sodium and potassium alkoxides such as sodium methoxide, sodium ethoxide and potassium tert-butoxide. The amount of base used is between 0.01 and 1.5 equivalents, more preferably between 0.05 and 0.20 equivalents.

The reaction between compound (V) and the dialkyl carbonate is preferably carried out in the presence of a solvent. Suitable solvents include, but are not limited to toluene, dimethyl carbonate, diethyl carbonate and dioxane.

The reaction can be carried out at a temperature from −10° C. to 150° C., preferably from 70° C. to 120° C.

Amino alcohols of formula (V), when not commercially available, may be made by a variety of literature routes such as shown below and as detailed in J. March, Advanced Organic Chemistry, 4^{th} ed. Wiley, New York 1992.

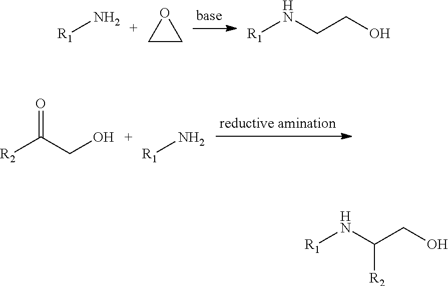

The compounds used in the process of the invention may exist as different geometric isomers, or in different tautomeric forms. This invention covers the production of all such isomers and tautomers, and mixtures thereof in all proportions, as well as isotopic forms such as deuterated compounds.

The compounds used in the process of this invention may also contain one or more asymmetric centers and may thus give rise to optical isomers and diastereomers. While shown without respect to stereochemistry, the present invention includes all such optical isomers and diastereomers as well as the racemic and resolved, enantiomerically pure R and S stereoisomers and other mixtures of the R and S stereoisomers and agrochemically acceptable salts thereof. It is recognized certain optical isomers or diastereomers may have favorable properties over the other. Thus when disclosing and claiming the invention, when a racemic mixture is disclosed, it is clearly contemplated that both optical isomers, including diastereomers, substantially free of the other, are disclosed and claimed as well.

Alkyl, as used herein, refers to an aliphatic hydrocarbon chain and includes straight and branched chains e. g. of 1 to 6 carbon atoms such as methyl, ethyl, n-propyl, isopropyl, n-butyl, isobutyl, sec-butyl, t-butyl, n-pentyl, isopentyl, neo-pentyl, n-hexyl, and isohexyl.

Halogen, halide and halo, as used herein, refer to iodine, bromine, chlorine and fluorine.

Haloalkyl, as used herein, refers to an alkyl group as defined above wherein at least one hydrogen atom has been replaced with a halogen atom as defined above. Preferred haloalkyl groups are dihaloalkyl and trihaloalkyl groups. Examples of haloalkyl groups include chloromethyl, dichloromethyl, trichloromethyl, fluoromethyl, difluoromethyl and trifluoromethyl. Preferred haloalkyl groups are fluoroalkyl groups, especially diflluoroalkyl and trifluoroalkyl groups, for example, difluoromethyl and trifluoromethyl.

Cycloalkyl, as used herein, refers to a cyclic, saturated hydrocarbon group having from 3 to 6 ring carbon atoms. Examples of cycloalkyl groups are cyclopropyl, cyclobutyl, cyclopentyl and cyclohexyl.

Alkoxy, as used herein, refers to the group —OR, wherein R is an alkyl group as defined herein.

Nitro, as used herein, refers to the group —NO₂.

Aryl, as used herein, refers to an unsaturated aromatic carbocyclic group of from 6 to 10 carbon atoms having a single ring (e. g., phenyl) or multiple condensed (fused) rings, at least one of which is aromatic (e.g., indanyl, naphthyl). Preferred aryl groups include phenyl, naphthyl and the like. Most preferably, an aryl group is a phenyl group.

The present invention also provides novel intermediates of formula (IVa)

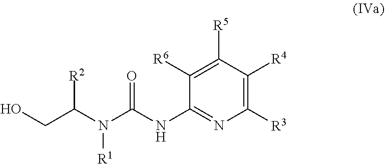

wherein
R¹ and R² are as defined above;
(i) one of R³, R⁴, R⁵ or R⁶ is C₁-C₆ haloalkyl and the other three are hydrogen;
(ii) R⁴ or R⁵ is halo, the other is hydrogen and R³ and R⁶ are both hydrogen; or
(iii) R⁵ is C₁-C₄ alkyl and R³, R⁴ and R⁶ are all hydrogen.

When R² is not hydrogen the compound (IVa) could be either an R or S enantiomer or any mixture of the two.

Preferably, the novel intermediates are selected from the group comprising:

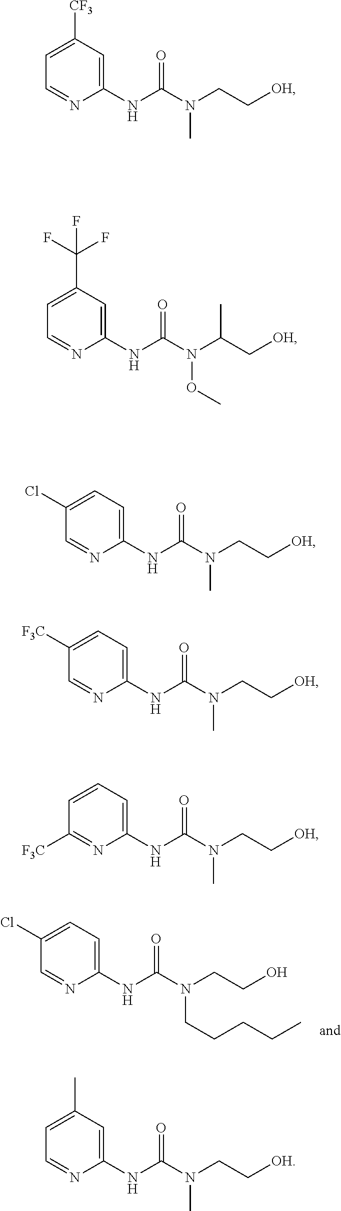

Additionally one specific form of the intermediate compound of formula (III) is novel. As such, the present invention also provides a novel intermediate of formula (IIIa):

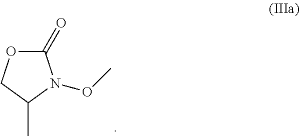

Compound (IIIa) could be either an R or S enantiomer or any mixture of the two.

Various aspects and embodiments of the present invention will now be illustrated in more detail by way of example. It will be appreciated that modification of detail may be made without departing from the scope of the invention.

For the avoidance of doubt, where a literary reference, patent application, or patent, is cited within the text of this application, the entire text of said citation is herein incorporated by reference.

### EXAMPLES

The following abbreviations were used in this section: s=singlet; bs=broad singlet; d=doublet; dd=double doublet; dt=double triplet; t=triplet, tt=triple triplet, q=quartet, sept=septet; m=multiplet; RT=retention time, MH⁺=molecular mass of the molecular cation.

¹H NMR spectra were recorded on a Bruker Avance III 400 spectrometer equipped with a BBFOplus probe at 400 MHz.

### Example 1: Preparation of 1-(2-hydroxyethyl)-1-methyl-3-[4-(trifluoromethyl)-2-pyridyl]urea

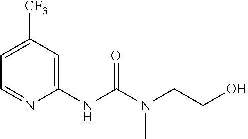 [SMILES: CN(CCO)C(=O)Nc1cc(C(F)(F)F)ccn1]

To a mixture of 2-amino-4-(trifluoromethyl)-pyridine (5.00 g, 29.9 mmol) and sodium tert-butoxide (4.40 g, 44.9 mmol) was added dry toluene (22 ml). After stirring the resulting mixture for 5 min 3-methyl-1,3-oxazolidin-2-one (9.26 g, 89.8 mmol) was added. The resulting black solution was stirred for 3.5 h at ambient temperature. Towards the end the reaction mixture changed to a brown thick suspension. The reaction was quenched by addition of water and diluted with ethyl acetate. Phases were separated and the aqueous phase was extracted with EtOAc (2×). The combined organic layers were washed with brine and dried over anhydrous Na₂SO₄. Evaporation under reduced pressure afforded 1-(2-hydroxyethyl)-1-methyl-3-[4-(trifluoromethyl)-2-pyridyl]urea (10.63 g) as a brown solid. Quantitative NMR analysis using trimethoxybenzene as an internal standard indicated purity of 72% (97% chemical yield). Thus obtained material was recrystallized from EtOAc (50 ml) to provide 1-(2-hydroxyethyl)-1-methyl-3-[4-(trifluoromethyl)-2-pyridyl]urea (5.90 g, 75%, >99% purity) as a white crystalline solid.

¹H NMR (400 MHz, CDCl₃) δ 8.99 (br, 1H), 8.30 (d, J=5.1 Hz, 1H), 8.25 (s, 1H), 7.11 (dd,J=5.3, 0.9 Hz, 1H), 4.39 (br, 1H), 3.90-3.84 (m, 2H), 3.55-3.50 (m, 2H), 3.03 (s, 3H); ¹⁹F NMR (400 MHz, CDCl₃) δ −64.96.

Alternatively, the same compound can be also obtained by carrying out the following procedure:

To a suspension of NaNH₂ (0.092 g, 2.24 mmol) in dry THF (1.2 ml) was added a solution of 3-methyl-1,3-oxazolidin-2-one (0.309 g, 2.99 mmol) and 2-amino-4-(trifluoromethyl)-pyridine (0.250 g, 1.50 mmol) in a dry THF (1.0 ml) at 0° C. The resulting dark solution was stirred at 0 C for 30 min and at ambient temperature for 5 h. A beige suspension had formed at the end of the reaction. The reaction was quenched by addition of acetic acid (0.27 ml, 4.8 mmol), diluted with methylene chloride and the remaining precipitate was filtered off. The filtrate was evaporated under reduced pressure and dissolved in methylene chloride (10 ml). This solution was washed with aq saturated NaHCO₃, aq saturated NH₄Cl, water (2×) and brine. The remaining organic phase was evaporated of afford 1-(2-hydroxyethyl)-1-methyl-3-[4-(trifluoromethyl)-2-pyridyl]urea (0.324 g) as a beige solid. Quantitative NMR analysis using trimethoxybenzene as an internal standard indicated purity of 89% (73% chemical yield).

### Example 2: Preparation of (2S)-2-(methoxyamino)propan-1-ol

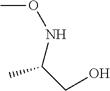

To a suspension of lithium aluminum hydride (3.34 g, 87.9 mmol) in dry THF (200 ml) was added at 0° C. dropwise over 20 min a solution of (2S)-2-(methoxyamino)propanoate (15.0 g, 78% purity, 87.9 mmol) in dry THF (25 ml). The reaction mixture was stirred for 1 h and allowed to warm to ambient temperature (full conversion). The reaction mixture was cooled to 0° C. and water (4.28 ml) was slowly added followed by 15% aq NaOH (4.28 ml) and another portion of water (12.84 ml) while keeping the temperature below 5° C. The resulting mixture was stirred at ambient temperature for 30 min, diluted with THF (100 ml) and filtered through a pad of celite. The filtrate was dried over anhydrous Na₂SO₄ and evaporated under reduced pressure to afford crude material (10.40 g). A short path distillation (0.06 mbar, 36° C.) provided (2S)-2-(methoxyamino)propan-1-ol (6.32 g, 96% purity, 66% yield) as a colourless liquid.

Analytical data matches those reported in WO 2010/106071

### Example 3: Preparation of (4S)-3-methoxy-4-methyl-oxazolidin-2-one

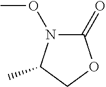

To a solution of (2S)-2-(methoxyamino)propan-1-ol (1.00 g, 88% purity, 8.37 mmol) in dry toluene (8.4 ml) was added diethyl carbonate (2.0 ml, 16.7 mmol) followed by KOtBu (0.094 g, 0.837 mmol). The resulting reaction mixture was heated at reflux for 19 h. The reaction mixture was cooled to ambient temperature, diluted with EtOAc and quenched with 1M HCl. Phases were separated and organic phase was washed with water and brine. Organic layer was dried over anhydrous Na₂SO₄ and evaporated under reduced pressure to provide a crude material (0.94 g). Purification by silica gel chromatography (0-30% EtOAc in cyclohexane) afforded (4S)-3-methoxy-4-methyl-oxazolidin-2-one (0.720 g, 93% purity, 61% yield) as a colourless liquid.

¹H NMR (400 MHz, CDCl₃) δ 4.33 (dd, J=8.1, 7.0 Hz, 1H), 3.97-3.88 (m, 1H), 3.88-3.82 (m, 4H), 1.37 (d, J=6.2 Hz, 3H); ¹³C NMR (100 MHz, CDCl₃) δ 158.8, 67.5, 64.0, 54.5, 15.8.

### Example 4: Preparation of 1-[(1S)-2-hydroxy-1-methyl-ethyl]-1-methoxy-3-[4-(trifluoromethyl)-2-pyridyl]urea

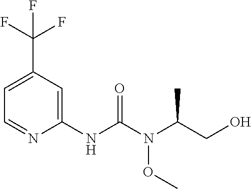 [SMILES: CON(C(=O)Nc1cc(C(F)(F)F)ccn1)[C@@H](C)CO]

2-Amino-4-(trifluoromethyl)pyridine (6.576 g, 39.3 mmol) was dissolved in dry THF (26 ml) and the solution was cooled to −5 C. 2.0M NaOtBu in THF (19.7 ml, 39.3 mmol) was added over 10 min. After stirring at this temperature for 1 h a solution of (4S)-3-methoxy-4-methyl-oxazolidin-2-one (4.00 g, 26.23 mmol) in THF (4 ml) was added and stirring was continued for 1 h 15 min. The reaction mixture was quenched with 2M HCl to pH 3. The resulting mixture was extracted with DCM (3×), combined organic layers were washed with brine and dried over anhydrous Na₂SO₄. Evaporation under reduced pressure provided 1-[(1S)-2-hydroxy-1-methyl-ethyl]-1-methoxy-3-[4-(trifluoromethyl)-2-pyridyl]urea (8.26 g, 86% purity, 92% chemical yield) as an orange oil which crystallized upon standing.

¹H NMR (400 MHz, CD₃OD) δ 8.49 (d, J=5.1 Hz, 1H), 8.36-8.33 (m, 1H), 7.33 (dd, J=5.1, 1.1 Hz, 1H), 4.41-4.31 (m, 1H), 3.86 (s, 3H), 3.75 (dd, J=11.2, 8.6 Hz, 1H), 3.58 (dd, J=11.4, 5.5 Hz, 1H), 1.22 (d, J=7.0 Hz, 3H); ¹⁹F NMR (400 MHz, CDCl₃) δ −66.57.

### Example 5: Preparation of 4-hydroxy-1-methyl-3-[4-(trifluoromethyl)-2-pyridyl]imidazolidin-2-one

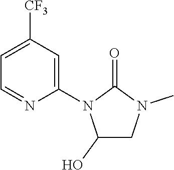 [SMILES: CN1CC(O)N(c2cc(C(F)(F)F)ccn2)C1=O]

To a solution of 1-(2-hydroxyethyl)-1-methyl-3-[4-(trifluoromethyl)-2-pyridyl]urea (10.0 g, 36.1 mmol) in EtOAc (300 ml) was added NaBr (0.375 g, 3.60 mmol) and 4-acetylamino-TEMPO (0.393 g, 1.80 mmol). The resulting solution was cooled to 0° C. and 5% aqueous solution of NaOCl (54 ml, 39.7 mmol) adjusted to pH 9.5 by NaHCO₃ (0.6 g) was added over 15 min. The color of the reaction mixture changed from pale yellow to orange. After stirring at 0° C. for 30 min another portion of 5% aq NaOCl (9.8 ml, 7.20 mmol) was added and the reaction was stirred for further 30 min. At this stage starting material was fully consumed. The reaction mixture was diluted with water, phases were separated and aqueous layer was extracted with EtOAc (3×200 ml). The combined organic layers were washed with brine, dried over anhydrous Na₂SO₄ and evaporated under reduced pressure to afford crude material (10.0 g). This material was suspended in n-hexane (100 ml) and heated to 70 C. TBME (80 ml) was added and heating was continued for 30 min. The remaining solid was filtered off and the filtrate was slowly cooled to 0° C. The resulting precipitate was filtered, washed on filter with n-hexane and dried under high vacuum to afford 4-hydroxy-1-methyl-3-[4-(trifluoromethyl)-2-pyridyl]imidazolidin-2-one (7.4 g, 75%) as a white solid.

Analytical data matches those reported in WO 2015/059262

### Example 6: Preparation of (5S)-4-hydroxy-1-methoxy-5-methyl-3-[4-(trifluoromethyl)-2-pyridyl]imidazolidin-2-one

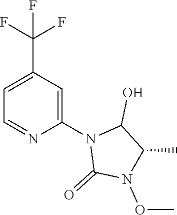 [SMILES: CON1C(=O)N(c2cc(C(F)(F)F)ccn2)C(O)[C@@H]1C]

To a solution of 1-[(1S)-2-hydroxy-1-methyl-ethyl]-1-methoxy-3-[4-(trifluoromethyl)-2-pyridyl]urea (10.0 g, 96% purity, 32.7 mmol) in ethyl acetate (300 ml) was added NaBr (0.337 g, 3.27 mmol) and 4-acetamido-2,2,6,6-tetramethylpiperidino-1-oxyl (0.356 g, 1.64 mmol). The resulting suspension was cooled to 0° C. An aqueous solution of NaClO (5.0%, 57.8 ml, 36.0 mmol) adjusted to pH 9.5 by addition of NaHCO₃ (1.05 g) was added over 10 min. After stirring for another 10 min (full conversion) the layers were separated, the organic layer was washed with water (2×) and brine and dried over anhydrous Na₂SO₄. Evaporation under reduced pressure provided crude material (10.01 g) which was purified by trituration with n-pentane (2×20 ml) to afford (5S)-4-hydroxy-1-methoxy-5-methyl-3-[4-(trifluoromethyl)-2-pyridyl]imidazolidin-2-one (7.82 g, 95% purity, 78% yield) as an off white solid.

Analytical data matches those reported in WO 2015/052076

### Example 7: Preparation of 3-(5-chloro-2-pyridyl)-1-(2-hydroxyethyl)-1-methyl-urea

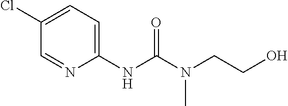

Sodium hydride (60% in paraffin oil, 0.114 g, 2.86 mmol) was washed twice under Ar with n-hexane (2 ml). A solution of 2-amino-5-chloropyridine (0.250 g, 1.91 mmol) in 2-MeTHF (2.5 ml) was added slowly. The grey-green suspension was stirred until no more gas evolution was observed and then 3-methyl-2-oxazolidinone (0.393 g, 3.81 mmol) was added. The resulting reaction mixture was stirred at room temperature for 20 h. The reaction was quenched by careful addition of water and diluted with EtOAc. Phases were separated and aqueous phase was extracted with EtOAc (2×). The combined organic layers were washed with brine, dried over anhydrous Na₂SO₄ and evaporated under reduced pressure to afford a crude residue (0.428 g). Quantitative ¹H NMR analysis using trimethoxy benzene as an internal standard indicated purity of 71% (69% chemical yield). The crude product was purified by silica gel chromatography (eluting with 1-4% MeOH in DCM) to afford 3-(5-chloro-2-pyridyl)-1-(2-hydroxyethyl)-1-methyl-urea (0.233 g, 95% purity, 50%) as a white solid.

¹H NMR (400 MHz, d₆DMSO) δ 9.21 (br, 1H), 8.22 (dd, J=2.6, 0.7 Hz, 1H), 7.83-7.80 (m, 1H), 7.79-7.75 (m, 1H), 5.35 (br, 1H), 3.59 (q, J=5.1 Hz, 2H), 3.43-3.36 (m, 2H), 2.94 (s, 3H).

### Example 8: Preparation of 1-(2-hydroxyethyl)-1-methyl-3-[5-(trifluoromethyl)-2-pyridyl]urea

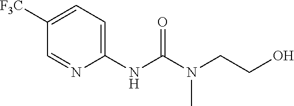 [SMILES: CN(CCO)C(=O)Nc1ccc(C(F)(F)F)cn1]

Sodium hydride (60% in paraffin oil, 0.0907 g, 2.27 mmol) was washed twice under Ar with n-hexane (2 ml). A solution of 2-amino-5-chloropyridine (0.250 g, 1.51 mmol) in 2-MeTHF (2.0 ml) was added slowly. The brown-red suspension was stirred until no more gas evolution was observed and then 3-methyl-2-oxazolidinone (0.312 g, 3.02 mmol) was added. The resulting reaction mixture was stirred at room temperature for 20 h. The reaction was quenched by careful addition of water and diluted with EtOAc. Phases were separated and aqueous phase was extracted with EtOAc (2×). The combined organic layers were washed with brine, dried over anhydrous Na₂SO₄ and evaporated under reduced pressure to afford a crude residue (0.457 g). Quantitative ¹H NMR analysis using trimethoxy benzene as an internal standard indicated purity of 45% (52% chemical yield). The crude product was purified by silica gel chromatography (eluting with 1-4% MeOH in DCM) to afford 1-(2-hydroxyethyl)-1-methyl-3-[5-(trifluoromethyl)-2-pyridyl]urea (0.177 g, 99% purity, 44%) as a pale yellow solid.

¹H NMR (400 MHz, d₆DMSO) δ 9.56 (br, 1H), 8.56 (dd, J=1.5, 0.7 Hz, 1H), 8.03 (dd, J=9.0, 2.6 Hz, 1H), 7.97-7.93 (m, 1H), 5.42 (br, 1H), 3.62 (q, J=4.9 Hz, 2H), 3.46-3.38 (m, 2H), 2.96 (s, 3H).

### Example 9: Preparation of 1-(2-hydroxyethyl)-1-methyl-3-(2-pyridyl)urea

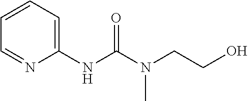

To a solution of 2-amino pyridine (0.250 g, 2.63 mmol) in dry toluene (2.0 ml) was added 2.0M NaOtBu in THF (2.63 mmol, 5.26 mmol). After stirring for 5 min 3-methyl-2-oxazolidinone (1.36 g, 13.1 mmol) was added and the resulting solution was stirred at ambient temperature for 23 h. The reaction mixture was quenched by addition of water and diluted with EtOAc. The phases were separated and the aqueous layer was extracted with EtOAc (2×). The combined organic layers were washed with water and brine and dried over anhydrous Na₂SO₄. Evaporation under reduced pressure afforded crude 1-(2-hydroxyethyl)-1-methyl-3-(2-pyridyl)urea (0.849 g) as a yellow liquid. Quantitative ¹H NMR analysis using trimethoxy benzene as an internal standard indicated purity of 39% (65% chemical yield).

¹H NMR (400 MHz, CDCl₃) δ 8.68 (br, 1H), 8.14-8.10 (m, 1H), 7.92-7.88 (m, 1H), 7.60 (ddd, J=8.7, 7.1, 2.2 Hz, 1H), 6.87 (ddd, J=7.3, 5.1, 1.1 Hz, 1H), 3.84-3.79 (m, 2H), 3.50-3.46 (m, 2H), 3.00 (s, 3H).

### Example 10: Preparation of 1-(2-hydroxyethyl)-3-[6-(trifluoromethyl)-2-pyridyl]urea

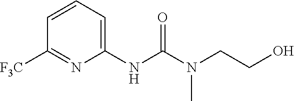 [SMILES: CN(CCO)C(=O)Nc1cccc(C(F)(F)F)n1]

Sodium hydride (60% in paraffin oil, 0.0886 g, 2.31 mmol) was washed twice under Ar with n-hexane (2 ml). A solution of 2-amino-5-chloropyridine (0.250 g, 1.54 mmol) in 2-MeTHF (2.0 ml) was added slowly. The gray suspension was stirred until no more gas evolution was observed and then 3-methyl-2-oxazolidinone (0.318 g, 3.08 mmol) was added. The resulting reaction mixture was stirred at room temperature for 20 h. The reaction was quenched by careful addition of water and diluted with EtOAc. Phases were separated and aqueous phase was extracted with EtOAc (2×). The combined organic layers were washed with brine, dried over anhydrous Na₂SO₄ and evaporated under reduced pressure to afford a crude residue (0.432 g). Quantitative ¹H NMR analysis using trimethoxy benzene as an internal standard indicated purity of 42% (48% chemical yield). The crude product was purified by silica gel chromatography (eluting with 1-4% MeOH in DCM) to afford 1-(2-hydroxyethyl)-3-[6-(trifluoromethyl)-2-pyridyl]urea (0.190 g, 97% purity, 45%) as a white solid.

¹H NMR (400 MHz, CDCl₃) δ 8.18 (d, J=8.4 Hz, 1H), 8.14 (br, 1H), 7.78 (t, J=8.1 Hz, 1H), 7.29 (d, J=7.7 Hz, 1H), 3.91-3.83 (m, 2H), 3.59-3.53 (m, 2H), 3.09 (s, 3H), 3.05 (br, 1H).

### Example 11: Preparation of 3-(5-chloro-2-pyridyl)-1-(2-hydroxyethyl)-1-pentyl-urea

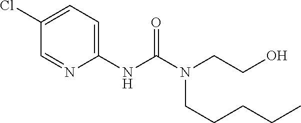 [SMILES: CCCCCN(CCO)C(=O)Nc1ccc(Cl)cn1]

Sodium hydride (60% in paraffin oil, 0.110 g, 2.86 mmol) was washed with n-hexane (2 ml) under Ar. A solution of 2-amino-5-chloropyridine (0.25 g, 1.91 mmol) in 2-MeTHF (2.5 ml) was added slowly. The resulting grey-green suspension was stirred for 30 min at ambient temperature and then 3-pentyloxazolidin-2-one (0.655 g, 3.81 mmol) was added. The resulting brown suspension was stirred at room temperature for 4 h before being quenched by addition of water. EtOAc was added, phases were separated and aqueous phase was extracted with EtOAc (2×). The combined organic layers were washed with brine and dried over anhydrous Na₂SO₄. Evaporation under reduced pressure afforded the crude product (0.793 g) as a brown liquid. Purification by silica gel chromatography (1-4% MeOH in DCM) afforded 3-(5-chloro-2-pyridyl)-1-(2-hydroxyethyl)-1-pentyl-urea (0.224 g, 89.5% purity, 37% yield) as a yellow solid.

¹H NMR (400 MHz, CDCl₃) δ 9.08 (br, 1H), 8.08 (d, J=2.2 Hz, 1H), 7.94 (d, J=8.8 Hz, 1H), 7.58 (dd, J=8.8, 2.6 Hz, 1H), 4.83 (br, 1H), 3.85 (t, J=4.6 Hz, 2H), 3.49 (t, J=4.6 Hz, 1H), 3.34-3.23 (m, 2H), 1.67-1.54 (m, 2H), 1.40-1.24 (m, 4H), 0.90 (t, J=7.0 Hz, 3H).

### Example 12: Preparation of 1-(2-hydroxyethyl)-1-methyl-3-(4-methyl-2-pyridyl)urea

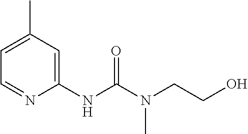

To a solution of 2-amino-4-methylpyridine (0.250 g, 2.29 mmol) in THF (3 ml) at 0 C was added a solution of sodium bis(trimethylsilyl)amine in THF (1.0M, 3.4 ml, 3.4 mmol). After stirring for 26 h at ambient temperature the reaction mixture was quenched by addition of water. The resulting mixture was taken up in EtOAc. Phases were separated and aqueous layer was extracted with EtOAc (2×). The combined organic layers were washed with brine and dried over anhydrous Na₂SO₄. Evaporation under reduced pressure provided a crude residue (0.414 g) as a brown oil. Quantitative ¹H NMR analysis using trimethoxy benzene as an internal standard indicated purity of 48% (41% chemical yield). Analytically pure sample (pale yellow solid) was obtained by reverse phase HPLC (eluting with 5-20% MeCN in water).

¹H NMR (400 MHz, CDCl₃) δ 8.88 (br, 1H), 8.01-7.95 (m, 2H), 6.81 (dd, J=5.3, 0.9 Hz, 1H), 3.90-3.85 (m, 2H), 3.62-3.56 (m, 2H), 3.07 (s, 3H), 2.38 (s, 3H).

1. A process for the preparation of compound of formula (I) 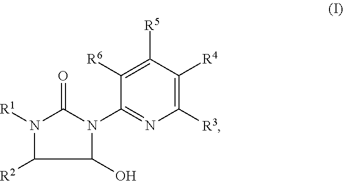
wherein
R¹ is selected from C₁-C₆ alkyl, C₃-C₆ cycloalkyl, C₁-C₆ alkoxy and aryl;
R² is selected from C₁-C₆ alkyl, aryl and hydrogen
R³, R⁴, R⁵ and R⁶ are each independently selected from hydrogen, C₁-C₆ alkyl, C₁-C₆ haloalkyl, nitro and halogen; comprising
a) reacting the compound of formula (II) 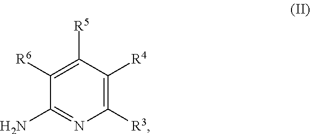
wherein R³, R⁴, R⁵ and R⁶ are as defined above with a strong base and a compound of formula (III) 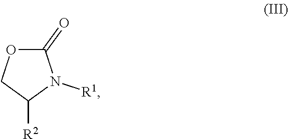
wherein R¹ and R² are as defined above to a compound of formula (IV) 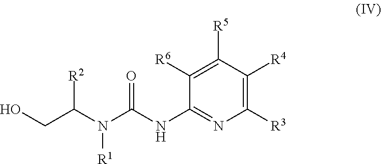
wherein R¹, R², R³, R⁴, R⁵ and R⁶ are as defined above, and
b) reacting the compound of formula (IV) with an oxidizing agent to produce a compound of formula (I) 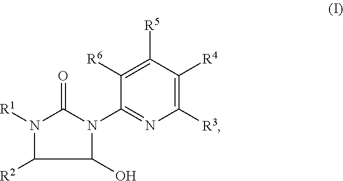
wherein
R¹, R², R³, R⁴, R⁵ and R⁶ are as defined above.

2. The process of claim 1, wherein the base is an alkali metal alkoxide, an alkali metal amide, an organolithium reagent or sodium hydride.

3. The process of claim 1, wherein step (a) is carried out in the presence of a solvent.

4. The process of claim 3, wherein the solvent is a non-protic organic solvent.

5. The process of claim 1, wherein step (a) is carried out at a temperature from −20° C. to 100° C.

6. The process of claim 1, wherein the oxidizing agent is aqueous sodium hypochlorite, oxygen, Dess-Martin periodinane or dimethylsulfoxide in the presence of an activating agent.

7. The process of claim 1, wherein step (b) is carried out in the presence of a solvent.

8. The process of claim 7, wherein the solvents is a polar non-water miscible solvent.

9. The process of claim 1, wherein step (b) is carried out at a temperature from −10° C. to 100° C.

10. The process of claim 1, wherein the compound of formula (III) is prepared by reacting an amino alcohol of formula (V) 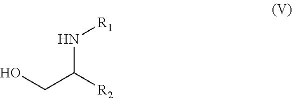
wherein R¹ and R² are as defined in claim 1 with a dialkyl carbonate in the presence of base.

11. The process of claim 10, wherein the dialkyl carbonate is dimethyl carbonate or diethyl carbonate.

12. The process of claim 10, wherein the base is a sodium or potassium alkoxide.

13. The process of claim 10, which is carried out in the presence of a solvent.

14. The process of claim 13, wherein the solvent is toluene, dimethyl carbonate, diethyl carbonate or dioxane.

15. The process of claim 10, which is carried out at a temperature from −10° C. to 150° C.

16. The process of claim 1, wherein R¹ is selected from C₁-C₅ alkyl and C₁-C₅ alkoxy.

17. The process of claim 16, wherein R¹ is selected from methyl and methoxy.

18. The process of claim 1, wherein R² is selected from hydrogen and C₁-C₅ alkyl.

19. The process of claim 18, wherein R² is selected from methyl and hydrogen.

20. The process of claim 1, wherein R³ is selected from hydrogen, C₁-C₄ alkyl, C₁-C₄ haloalkyl and halo.

21. The process of claim 20, wherein R³ is selected from hydrogen, chloro, methyl, difluoromethyl and trifluoromethyl.

22. The process of claim 1, wherein R⁴ is selected from hydrogen, C₁-C₄ alkyl, C₁-C₄ haloalkyl and halo.

23. The process of claim 22, wherein R⁴ is selected from hydrogen, chloro, methyl, difluoromethyl and trifluoromethyl.

24. The process of claim 1, wherein R⁵ is selected from hydrogen, C₁-C₄ alkyl, C₁-C₄ haloalkyl and halo.

25. The process of claim 24, wherein R⁵ is selected from hydrogen, chloro, methyl, difluoromethyl and trifluoromethyl.

26. The process of claim 1, wherein R⁶ is selected from hydrogen, C₁-C₄ alkyl, C₁-C₄ haloalkyl and halo.

27. The process of claim 26, wherein R⁶ is selected from hydrogen, chloro, methyl, difluoromethyl and trifluoromethyl.

28. A compound of formula (IVa) 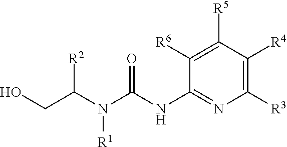
wherein
R¹, R² are as defined above;
(i) one of R³, R⁴, R⁵ or R⁶ is C₁-C₆ haloalkyl and the other three are hydrogen;
(ii) R⁴ or R⁵ is halo, the other is hydrogen and R³ and R⁶ are both hydrogen; or
(iii) R⁵ is C₁-C₄ alkyl and R³, R⁴ and R⁶ are both hydrogen.

29. A compound of formula (IIIa): [chemical structure]
